(19) United States
(12) Patent Application Publication
(10) Pub. No.: US 2012/0282340 A1
(43) Pub. Date: Nov. 8, 2012

(54) DRUG DELIVERY SYSTEM

(75) Inventors: Ildiko Terebesi, Berlin (DE); Adrian Funke, Berlin (DE); Konstanze Diefenbach, Beijing (CN); Matthias Schäfers, Berlin (DE); Sascha General, Berlin (DE)

(73) Assignee: Bayer Schering Pharma Aktiengesellschaft, Berlin (DE)

(21) Appl. No.: 13/130,326

(22) PCT Filed: Nov. 13, 2009
(86) PCT No.: PCT/EP2009/008080

§ 371 Date: Aug. 9, 2011

(30) Foreign Application Priority Data
Nov. 21, 2008 (EP) 08105842.2
Jul. 17, 2009 (EP) 09075321.1

(51) Int. Cl. A61K 9/00; A61P 5/24; A61K 31/565

(52) U.S. Cl. 424/485; 424/488; 424/486; 424/484; 514/182

(57) ABSTRACT

The present invention relates to drug delivery systems in the form of thin water-soluble films (wafers), which contain an Estrogen Receptor beta (ER-β) selective agonist. The wafers of the present invention are suitable for treating, alleviating or preventing a physical condition in a female mammal caused by insufficient endogenous levels of estrogen.

### FIELD OF THE INVENTION

The present invention relates to drug delivery systems in the form of thin water-soluble films (wafers), which contain an Estrogen Receptor β (ER-β) selective agonist, in particular a 8β- or 9α-substituted oestra-1,3,5(10)-triene as a Estrogen Receptor β (ER-β) selective agonist, specifically a ER-β selective agonist chosen from the compounds:
9α-Vinyl-estra-1,3,5(10)-triene-3,16α-diol,
17β-Fluoro-9α-vinyl-estra-1,3,5(10)-triene-3,16α-diol,
18a-Homo-9α-vinyl-estra-1,3,5(10)-triene-3,16α-diol,
16α-Fluor-8β-vinyl-estra-1,3,5(10)-triene-3,17α-diol,
8β-vinyl-16α-fluoro-estra-1,3,5(10)-triene-3,17β-diol,
16β-Fluoro-8β-vinyl-estra-1,3,5(10)-triene-3,17β-diol,
8β-vinyl-estra-1,3,5(10)-triene-3,17β-diol, or derivatives thereof.

The wafers of the present invention are suitable for treating, alleviating or preventing a physical condition in a female mammal caused by insufficient endogenous levels of estrogen.

### BACKGROUND OF THE INVENTION

While drugs, such as estrogens, may be included in traditional standard oral tablet or capsule formulations to provide an accurate and consistent dose, such delivery forms have several disadvantages in both the administration and preparation of the drug. For example, it has been estimated that about 50% of the population have problems swallowing tablets (see Seager in J. Pharmacol. Pharm. 1998; 50; 375-382), and patients such as children or the elderly who will not, or cannot, swallow tablets or capsules represent a challenge for the pharmaceutical industry. The pharmaceutical industry has tried to meet this challenge by developing a number of different drug delivery systems, including rapid in-mouth disintegrating tablets, tablets which disintegrate in liquid prior to ingestion, liquids and syrups, gums and even transdermal patches. However, each of these drug delivery systems can pose their own problems.

Transdermal patches can be inconvenient and uncomfortable as well as rather expensive to produce. Furthermore, the drug flux through the skin can also raise very complex dosing issues. Liquids are particularly useful for children. However, liquids can be inconvenient for adults and can be relatively expensive to formulate, package and transport. Tablets that can be dissolved in a liquid before ingestion can also be useful. However, they can also be quite inconvenient in that they require liquid and a drinking container to be provided. Furthermore, time is required for disintegration and/or dissolution, even when effervescent tablets are used. Finally, these drug delivery systems can be quite messy as they typically leave a particulate and/or scum in the glass. Rapid in-mouth disintegrating tablets, such as chewable or self disintegrating tablets offer great convenience. However, chewable or self-disintegrating tablets often present real taste masking problems as the act of chewing can disrupt protective coatings. Furthermore, chewable or self-disintegrating tablets are often associated with an unpleasant mouthfeel. Moreover, the fear of swallowing, chewing, or choking on such solid shaped articles is still a concern in certain populations. In addition, the fragility/friability of such porous, and low-pressure molded tablets makes them difficult to carry, store, handle and administer to patients, especially the children and the elderly.

Thus, there is a need for reliable delivery systems with improved patient compliance, i.e. where the dosing is easy and allows the patients to take their medications discretely wherever and whenever needed. Water-soluble films (wafers) fulfil those criteria. Usually, such wafers dissolve quickly in the saliva present in the mouth thereby releasing the active ingredient(s) which, in turn, can then be absorbed via the lingual, sublingual, buccal and/or the palatal route.

The pharmaceutical industry is constantly aiming at improving delivery systems in order to make a better utilisation of a given drug dose. Stated differently, there is a constant need for delivery systems where the drug load can be lowered while, at the same time, still give rise to clinical relevant concentrations of the drug in the blood stream. This is particularly relevant when high-potent drugs, such as steroid hormones, are to be administered. Lowering the dose of, e.g., a steroid hormone while still obtaining clinical relevant concentrations of the steroid hormone in the blood stream not only allows for savings in the pharmaceutical industry, as smaller amounts of drug is needed, but also allows for smaller total amounts of the steroid hormone to be administered to the patients. Evidently, this may lead to fewer cases of overdosing and less pronounced side-effects. The importance of administration of low doses of steroid hormones, such as estrogens, is also emphasised in the FDA guidelines where sponsors are encouraged to investigate dosing schedules and drug delivery systems that can achieve efficacy with the lowest possible exposures (Guidance for Industry: Estrogen and Estrogen/Progestin Drug Products to Treat Vasomotor Symptoms and Vulvar and Vaginal Atrophy Symptoms—Recommendations for Clinical Evaluation; U.S. Department of Health and Human Services; Food and Drug Administration; CDER; January 2003).

Yet another aim when creating new dosage forms for highly active drugs is to achieve low inter-individual variability with respect to the resulting serum levels of the drug in different patients in order to guarantee that comparable dosages of the drug will lead to comparable effects in different patients. This is particularly important if the therapeutic window of a given drug, i.e. the difference between the serum level of the drug that provides the desired therapeutic effect and the serum level that results in unacceptable adverse effects, is relatively small.

Chewable taste-masked pharmaceutical compositions are described in U.S. Pat. No. 4,800,087.

Taste-masked orally disintegrating tablets (ODTs) are described in US 2006/0105038.

Taste-masking coating systems are described in WO 00/30617.

Taste-masked wafers are described in WO 03/030883.

Selective estrogens represent a newer alternative to estrogen/progestogen combination products. Selective estrogens have to date been understood to be compounds having estrogen-like effects on brain, bone and vascular system because of their anti-uterotrophic (i.e. anti-estrogenic) partial effect, but not having a proliferative effect on the endometrium.

Estrogen receptor modulators with preference for ER-β, in particular ER-β selective agonists, may also have a beneficial effect on brain functions, bladder, intestine and the cardiovascular system without having in the same dose range a hepatic estrogen effect or stimulating effect on endometrium and breast. ER-β agonists therefore represent a novel option for selective estrogen therapy and for the treatment of hot flushes and mood fluctuations. The occurrence of hot flushes presumably derives from an instability of the hypothalamic thermoregulatory set point caused by the decline in estrogens and the onset of the menopause (Stearns V, Ullmer L, Loepez J F, Smith Y, Isaacs C, Hayes DF (2002) Hot flushes. The Lancet 360: 1851-1861).

The patent application WO 01/77139 A1 describes 8β-substituted estratrienes wherein R⁸ means a straight-chain or branched-chain, optionally partially or completely halogenated alkyl or alkenyl radical with up to 5 carbon atoms, an ethinyl- or prop-1-inyl radical, as pharmaceutical active ingredients that have in vitro a higher affinity to estrogen receptor preparations of rat prostates than to estrogen receptor preparations of rat uteri, their production, their therapeutic use and pharmaceutical dispensing forms that contain the said compounds

The patent document WO 03/104253A2 relates to novel 9 alpha-substituted estratrienes of general formula (I)—in which R⁹ represents a linear-chain or branched-chain, optionally partially or fully halogenated alkenyl radical comprising between 2 and 6 carbon atoms, or an ethinyl radical or a prop-1-inyl radical—as pharmaceutical active ingredients which have, in vitro, a higher affinity to estrogen receptor preparations of the rat prostate than to estrogen receptor preparation of the rat uterus, and, in vivo, preferably a preferential action on the ovary compared to the uterus.

The patent document PCT/EP2008/059115 refers to 8β-substituted estra-1,3,5(10)-triene derivatives of general formula I, their use as pharmaceutical active ingredients, which have in vitro a higher affinity to estrogen receptor preparations from rat prostates than to estrogen receptor preparations from rat uteri and in vivo a preferential action in the ovary in comparison to the uterus, their production, their therapeutic use and pharmaceutical dispensing forms that contain the new compounds.

Routine test for identifying the selective ER-β activity in vitro and in vivo are reported in the documents cited above, for example on page 18 to 23 of WO03/104253A2.

A further routine test to identify selective ER-β activity, is as follows:

Cellular in vitro assay to determine the estrogen receptor -α and -β activity

### ABBREVIATIONS

DMEM Dulbecco's modified Eagle medium
DNA deoxynucleic acid
FCS fetal calf serum
HEPES 4-(2-hydroxyethyl)-1-piperazineethanesulphonic acid
PCR polymerase chain reaction

Modulators of the human estrogen receptors -α and -β (ERα and ERβ) are identified, and the activity of the substances described herein is quantified, with theaid of recombinant cell lines. These cells are originally derived from a hamster ovary epithelial cell (Chinese Hamster Ovary, CH0 K1, ATCC: American Type Culture Collection, VA 20108, USA).

An established chimera system in which the ligand-binding domains of human steroid hormone receptors are fused to the DNA-binding domain of the yeast transcription factor GAL4 are used in this CH0 K1 cell line. The GAL4-steroid hormone receptor chimeras produced in this way are cotransfected and stably expressed with a reporter construct in the CHO cells.

### Cloning:

To generate the GAL4-steroid hormone receptor chimeras, the GAL4 DNA-binding domain (amino acids 1-147) from the vector pFC2-dbd (from stratagene) is cloned with the PCR-amplified ligand-binding domains of the estrogen receptor a (ERα, Genbank accession number NM00125, amino acids 282-595) and of the estrogen receptor β (ERβ, Genbank accession number AB006590, amino acids 223-530) into the vector pIRES2 (from Clontech). The reporter construct, which comprises five copies of the GAL4 binding site upstream of a thymidine kinase promoter, leads to expression of firefly luciferase (Photinus pyralis) after activation and binding of the GAL4-estrogen receptor chimeras by specific agonists.

Assay procedure: the stock cultures of ERα and ERβ cells are routinely cultured in DMEM/F12 medium, 10% FCS, 1% Hepes, 1% penicillin/streptomycin, 1 mg/ml G418, and 5 μg/ml puromycin. On the day before the assay, the ERα and ERβ cells are plated out in Opti-MEM medium (Optimem, from Invitrogen, 2.5% activated carbon-purified FCS from Hyclone, 1% Hepes) in 96- (or 384) well microtitre plates and kept in a cell incubator (96% humidity, 5% v/v CO₂, 37° C.). On the day of the assay, the substances to be tested are taken up in the abovementioned medium and added to the cells. If it is intended to investigate possible antagonistic properties of test substances, the estrogen receptor agonist 17-β oestradiol (from Sigma) is added 10 to 30 minutes after addition of the test substances, but no additional addition of 17-β oestradiol takes place in the investigation of agonistic properties. After a further incubation time of 5 to 6 hours, the cells are lysed with a Iuciferin/Triton buffer, and the luciferase activity is measured with the aid of a video camera. The measured relative light units as a function of the substance concentration result in a sigmoidal stimulation curve. The EC50 and values are calculated with the aid of the GraphPad PRISM (version 3.02) computer program.

The patent documents WO01/77139A1, WO03/104253A2 and PCT/EP2008/059115 are incorporated by reference in the present application.

### SUMMARY OF THE INVENTION

In a first aspect, the present invention relates to a unit dosage form comprising a thin water-soluble film matrix, wherein
a) said film matrix comprises at least one water-soluble matrix polymer;
b) said film matrix comprises a ER-β selective agonist, or a derivative thereof; and
c) said film matrix has a thickness of less than 300 μm.

In a further aspect, the present invention relates to a unit dosage form comprising a thin water-soluble film matrix, wherein
a) said film matrix comprises at least one water-soluble matrix polymer;
b) said film matrix comprises 8β- or 9α-substituted estra-1,3,5(10)-triene chosen from the group of compounds:
9α-Vinyl-estra-1,3,5(10)-triene-3,16α-diol,
17β-Fluoro-9α-vinyl-estra-1,3,5(10)-triene-3,16α-diol,
18a-Homo-9α-vinyl-estra-1,3,5(10)-triene-3,16α-diol,
16α-Fluor-8β-vinyl-estra-1,3,5(10)-triene-3,17α-diol,
8β-vinyl-16α-fluoro-estra-1,3,5(10)-triene-3,17β-diol,
16β-Fluoro-8β-vinyl-estra-1,3,5(10)-triene-3,17β-diol,
8β-vinyl-estra-1,3,5(10)-triene-3,17β-diol, or derivatives thereof; and
   c) said film matrix has a thickness of less than 300 μm.

According to a further aspect of the present invention said film matrix comprises 5-5000 μg, specifically 10-3000 μg, more specifically such as 25-1500 μg of a 8β- or 9α-substituted estra-1,3,5(10)-triene chosen from the group of compounds:
9α-Vinyl-estra-1,3,5(10)-triene-3,16α-diol,
17β-Fluoro-9α-vinyl-estra-1,3,5(10)-triene-3,16α-diol,
18a-Homo-9α-vinyl-estra-1,3,5(10)-triene-3,16α-diol,
16α-Fluor-8β-vinyl-estra-1,3,5(10)-triene-3,17α-diol,
8β-vinyl-16α-fluoro-estra-1,3,5(10)-triene-3,17β-diol,
16β-Fluoro-8β-vinyl-estra-1,3,5(10)-triene-3,17β-diol,
8β-vinyl-estra-1,3,5(10)-triene-3,17β-diol, or derivatives thereof.

In another aspect, the present invention relates to a unit dosage form of the invention for use as a medicament.

In a further aspect, the present invention relates to a unit dosage form of the invention for treating, alleviating or preventing a physical condition in a female mammal caused by insufficient endogenous levels of estrogen. Examples of such physical conditions include, but is not limited to vasomotor symptoms (particularly hot flashes and night sweats), symptoms of urogenital atrophy, sleep disorders, memory problems, anxiety, depression and other mood disorders, decrease in bone mineral density, osteoporosis, and increased risk or incidence of bone fracture.

The present invention further refers to a wafer with an improved acceptability, said acceptability being a feature of primary importance for drug delivery systems to be administered in long term treatment.

According to the present invention, a unit dosage form (in the form of a wafer) that contains a low dose of a 8β- or 9α-substituted estra-1,3,5(10)-triene mentioned as ER-β selective agonist can be administered to female mammals via the intra-oral route and still give rise to clinical relevant serum levels in the blood stream.

The administered dose and the inter-individual variability with respect to the resulting serum levels of 8β- or 9α-substituted oestra-1,3,5(10)-triene can be lowered significantly compared to oral administration. The use of a ER-β selective agonist with reduced inter-individual variability of said active ingredient is particularly beneficial as it allows to avoid opposed treatment. An opposed treatment refers to a continuous or cyclic co-administration of a progestin which is commonly applied in conventional Hormone Replacement Therapy in order to counteract the estrogen-induced Estrogen Receptor alpha (ER-α) mediated proliferation of the endometrium. An opposed treatment is not an absolute requirement when using the wafer according to the present invention because the inter-individual variability with respect to serum levels is smaller than the selectivity of the wanted therapeutic ER-β mediated activity over the unwanted ER-α mediated endometrium-stimulating activity.

Interestingly, this favourable ratio of wanted versus unwanted effects cannot be realized with a traditional standard oral tablet formulation. Therefore, due to the considerable lowered dose and the reduced inter-individual variability with respect to serum levels allowing the omission of progestin co-administration, wafer formulations of 8β- or 9α-substituted oestra-1,3,5(10)-trienes are thought to deliver a superior safety profile compared to other application forms.

Low inter-individual variability with respect to the 8β- or 9α-substituted estra-1,3,5(10)-triene serum level and the resulting ER-β mediated therapeutic effect, would allow for administration of the same lowest effective dose in a high number of patients and is likely to substantially reduce the potential for unwanted adverse effects.

Still another object of the invention is to provide a unit dosage form which contains a lowered dose of 8β- or 9α-substituted oestra-1,3,5(10)-triene, particularly of 9α-Vinyl-estra-1,3,5(10)-triene-3,16α-diol, 17β-Fluoro-9α-vinyl-estra-1,3,5(10)-triene-3,16α-diol, 18a-Homo-9α-vinyl-estra-1,3,5(10)-triene-3,16α-diol, 16α-Fluor-8β-vinyl-estra-1,3,5(10)-triene-3,17α-diol, 8β-vinyl-16α-fluoro-estra-1,3,5(10)-triene-3,17β-diol, 16β-Fluoro-8β-vinyl-estra-1,3,5(10)-triene-3,17β-diol, 8β-vinyl-estra-1,3,5(10)-triene-3,17β-diol, or derivatives thereof, as compared to standard oral treatment, but which still give rise to a clinical relevant concentration of the 8β- or 9α-substituted oestra-1,3,5(10)-triene as active ingredient in the blood stream of the patient.

A further object of the invention is to provide a unit dosage form to be applied to the oral cavity which gives rise to fewer side effects, as compared to standard oral treatment, but which is still effective in treating, alleviating or preventing a physical condition in a female mammal caused by insufficient endogenous levels of estrogen.

The invention further refers to a unit dosage form to be applied to the oral cavity which is better tolerated with regard to gynaecological effects as compared to standard oral treatment, but which is still effective in treating, alleviating or preventing a physical condition in a female mammal caused by insufficient endogenous levels of estrogen.

The present invention provides a wafer with improved mouthfeel and taste, by defining favourable thickness and elasticity, able to confer improved patient acceptability.

The present invention provides further provides a unit dosage form to be applied to the oral cavity, which provides comparable average serum level as an oral formulation using a decreased dose, but where the patient compliance is higher.

Other aspects of the present invention will be apparent from the below description and the appended claims.

### DETAILED DESCRIPTION OF THE INVENTION

Herein, the term “active ingredient” is intended to mean any pharmaceutically active compound comprised in dosage form according to the present invention, and particularly a 8β- or 9α-substituted estra-1,3,5(10)-triene as ERβ agonist, more particularly a compound chosen from the group comprising:
9α-Vinyl-estra-1,3,5(10)-triene-3,16α-diol,
17β-Fluoro-9α-vinyl-estra-1,3,5(10)-triene-3,16α-diol,
18a-Homo-9α-vinyl-estra-1,3,5(10)-triene-3,16α-diol,
16α-Fluor-8β-vinyl-estra-1,3,5(10)-triene-3,17α-diol,
8β-vinyl-16α-fluoro-estra-1,3,5(10)-triene-3,17β-diol,
16β-Fluoro-8β-vinyl-estra-1,3,5(10)-triene-3,17β-diol,
8β-vinyl-estra-1,3,5(10)-triene-3,17β-diol, or derivatives thereof.
   Furthermore when used herein, the term “derivatives thereof” refers particularly to those esters of a 8β- or 9α-substituted estra-1,3,5(10)-triene which would be apparent to the pharmaceutical chemist, i.e. those which are substantially non-toxic and which may favourably affect the pharmacokinetic properties of the identified compounds, such as palatability, absorption, distribution, metabolism and excretion. Typically, an ester of the compounds related to the present invention is in the 3-position or 17-position of a 8β- or 9α-substituted oestra-1,3,5(10)-triene defined above. Specific examples of pharmaceutically acceptable esters include valerate, acetate, propionate, enantate, undecylate, benzoate, cypionate, sulfate and sulfamate esters.

The term “water-soluble film matrix”, wherein used herein, refers to a thin film which comprises, or consists of, a water-soluble polymer and active ingredients as well as other auxiliary components dissolved or dispersed in the water-soluble polymer. In a particular embodiment, at least an active ingredient is completely dissolved in the water-soluble polymer.

As used herein, the term “water-soluble polymer” refers to a polymer that is at least partially soluble in water, and particularly fully or predominantly soluble in water, or absorbs water. Polymers that absorb water are often referred to as being “water-swellable polymers”. The materials useful for the present invention may be water-soluble or water-swellable at room temperature (about 20° C.) and other temperatures, such as temperatures exceeding room temperature. Moreover, the materials may be water-soluble or water-swellable at pressures less than atmospheric pressure. The water-soluble polymers are water-soluble, or water-swellable having at least 20% by weight water uptake. Water-swellable polymers having 25% by weight, or more, water uptake, are also useful. The unit dosage forms of the present invention formed from such water-soluble polymers are in particular sufficiently water-soluble to be dissolvable upon contact with bodily fluids, in particular saliva. According to an embodiment of the invention, the water-soluble polymer is a mucoadhesive polymer. This will allow for transmucosal delivery of the active ingredient, particularly of the ER-β selective agonist, and ensure efficient uptake of the molecule by avoiding the first pass metabolism. The water-soluble polymer typically constitutes from 50-99.9% by weight, such as from 75-99% by weight, of the water-soluble film matrix.

The water-soluble matrix polymer (constituting the major part of the water-soluble film matrix) can be selected from the group consisting of a cellulosic material, a synthetic polymer, a gum, a protein, a starch, a glucan and mixtures thereof.

Examples of cellulosic materials suitable for the purposes described herein include carboxymethyl cellulose, methyl cellulose, ethyl cellulose, hydroxymethyl cellulose, hydroxyethyl cellulose, hydroxypropyl cellulose, hydroxymethylpropyl cellulose, hydroxypropylmethyl cellulose and combinations thereof. Particularly preferred cellulosic materials are hydroxypropylmethyl cellulose and hydroxy-propyl cellulose, in particular hydroxypropyl cellulose and hydroxypropylmethyl cellulose.

Examples of synthetic polymers include polymers commonly used as immediate-release (IR) coatings for pharmaceuticals, such as the PVA-PEG co-polymers, which are commercially available in different grades under the trademark Kollicoat® IR. Further examples of synthetic polymers include polyacrylic acid and polyacrylic acid derivatives

Examples of water-soluble gums include gum arable, xanthan gum, tragacanth, acacia, carageenan, guar gum, locust bean gum, pectin, alginates and combinations thereof.

Useful water-soluble protein polymers include gelatine, zein, gluten, soy protein, soy protein isolate, whey protein, whey protein isolate, casein, levin, collagen and combinations thereof.

Examples of useful starches include gelatinised, modified or unmodified starches. The source of the starches may vary and include pullulan, tapioca, rice, corn, potato, wheat and combinations thereof.

Additional water-soluble polymers, which may be used in accordance with the present invention, include dextrin, dextran and combinations thereof, as well as chitin, chitosin and combinations thereof, polydextrose and fructose oligomers.

In one embodiment of the invention the 8β- or 9α-substituted estra-1,3,5(10)-triene is 9α-Vinyl-estra-1,3,5(10)-triene-3,16α-diol, or derivatives thereof.

As indicated above, the unit dosage form of the invention comprises a low dose of an 8β- or 9α-substituted estra-1,3,5(10)-triene, particularly a compound chosen from the group comprising: 9α-Vinyl-estra-1,3,5(10)-triene-3,16α-diol, 17β-Fluoro-9α-vinyl-estra-1,3,5(10)-triene-3,16α-diol, 18a-Homo-9α-vinyl-estra-1,3,5(10)-triene-3,16α-diol, 16α-Fluor-8β-vinyl-estra-1,3,5(10)-triene-3,17α-diol, 8β-vinyl-16α-fluoro-estra-1,3,5(10)-triene-3,17β-diol, 16β-Fluoro-8β-vinyl-estra-1,3,5(10)-triene-3,17β-diol, 8β-vinyl-estra-1,3,5(10)-triene-3,17β-diol, or derivatives thereof, namely a dose of 5-5000 μg. In an interesting embodiment of the invention, the film matrix comprises 10-3000 μg of ER-β selective agonist, such as 25-1500 μg of ER-β selective agonist.

Examples of doses of ER-β selective agonist which may be comprised in the film matrix includes doses of about 10, 12.5, 15, 20, 25, 30, 40, 50, 60, 62.5, 70, 80, 90, 100, 120, 150, 180, 200, 250, 270, 300, 350, 360, 400, 450, 500, 540, 600, 625, 700, 800, 875, 900, 1000, 1100, 1250, 1500, 1750, 2000, 2500 or 3000 μg of ER-β selective agonist. Particular examples of doses of ER-β selective agonist which may be comprised in the film matrix are doses of about 15, 20, 25, 30, 40, 50, 60, 62.5, 70, 75, 80, 90, 100, 120, 150, 180, 200, 250, 270, 300, 500, 625, 875 or 1000 μg of ER-β selective agonist.

The above examples of doses refers in particular to the above specifically named compounds, and more particularly to 17β-Fluoro-9α-vinyl-estra-1,3,5(10)-triene-3,16α-diol.

The above-mentioned doses preferably correspond to the daily dose. It should be understood that the above-mentioned doses are indicated with respect to a ER-β selective agonist which is not esterified in position 3 or 7 of the steroidal skeleton. If a pharmaceutically acceptable ester of a ER-β selective agonist, is employed it will be understood that a dose which is therapeutically equivalent to the stated dose of the not esterified ER-β selective agonist should be used. It is routine for those skilled in the art to determine pharmacologically/therapeutically equivalent doses of such other forms when the effective dose of the ER-β selective agonist is known.

Stated differently, if a pharmaceutically acceptable ester of a ER-β selective agonist, is employed it will be understood that a dose which is equimolar to the stated dose of the not esterified ER-β selective agonist should be used, provided that the absorption of the not esterified ER-β selective agonist and the derivative thereof is the same, cf. below. Thus, a “therapeutically equivalent amount of the ER-β selective agonist derivative X” can be calculated by the following formula:

Dose_{ER-β agonist}×(MW_{ER-β derivative X}/MW_{ER-β agonist})

where MW indicates the molecular weight of the ER-β selective agonist in question. It will be understood that all of the above-indicated intervals and doses of ER-β selective agonist should be converted to the corresponding intervals and doses (using the above formula) if the ER-β selective agonist is used in its as a derivative. It will be understood, however, that the above formula can only be applied if the bioavailability and the Area Under the Curve (AUC) are identical for the ER-β selective agonist and the derivative in question. Thus, if the absorption of the ER-β agonist derivative in question differs from the absorption of the ER-β selective agonist, the amount of the ER-β agonist derivative required to achieve the average serum level of a given dose of the ER-β selective agonist is decisive for determining the therapeutically equivalent amount.

The paper of Timmer and Geurts provides guidance of how equivalent doses may be determined in the case of an estrogen (see “Bioequivalence assessment of three different estradiol formulations in postmenopausal women in an open, randomized, single-dose, 3-way cross-over” in European Journal of Drug Metabolism and Pharmacokinetics, 24(1):47-53,1999).

The unit dosage form of the invention is most preferably in the form of a thin film, which dissolves fast mainly due to the large surface area of the film, which wets quickly when exposed to the moist oral environment. Contrary to fast-dissolving tablets, which are usually soft, friable and/or brittle, the film is solid and strong, but still flexible and does not require special packaging. As indicated above, the film is thin and can be carried in the patient's pocket, wallet or pocket book.

The film may be applied under or on the tongue, to the upper palatine, to the inner cheeks or any oral mucosal tissue, of the female mammal. The film may be rectangular, oval, circular, or, if desired, a specific shape, cut to the shape of the tongue, the palatine or the inner cheeks, may be applied. The film is rapidly hydrated and will adhere onto the site of application. It then rapidly disintegrates and dissolves to release the ER-β selective agonist for oral mucosal absorption.

Concerning the dimensions of the unit dosage form of the invention, the water-soluble film forming matrix is formed into a dry film which typically has a thickness of less than 300 μm, in particular less than 250 μm, preferably less then 200 μm, such as less than 150 μm. More particularly, the thickness is less than 125 μm, such as less than 100 μm. Stated differently, the thickness is typically in the range of from 10-300 μm, in particular in the range of from 15-250 μm, particularly in the range of from 20-200 μm, such as in the range of from 25-150 μm. According to further embodiments of the invention, the thickness is in the range of from 25-125 μm, such as in the range of from 25-100 μm, e.g. in the range of from 30-90 μm, in particular in the range of from 40-80 μm. Specific, examples include thicknesses of about 30 μm, about 40 μm, about 50 μm, about 60 μm, about 70 μm, about 80 μm, about 90 μm or about 100 μm. Specific examples include thicknesses of about 40 μm, about 50 μm, about 60 μm, about 70 μm or about 80 μm.

The surface dimension (surface area) of the film matrix is typically in the range of from 2-10 cm², such as in the range of from 2-9 cm², e.g. in the range of from 2-8 cm², more particularly in the range of from 2-7 cm², in particular in the range of from 4-6 cm². Specific, and preferred, examples of the surface area include surface areas of about 3, 3.5, 4, 4.5, 5, 5.5, 6, 6.5 or 7 cm².

The total weight of the film matrix will typically be in the range of from 5-200 mg, such as in the range of from 5-150 mg, e.g. in the range of from 10-100 mg. More specifically, the total weight of the film matrix is in the range of from 10-75 mg, such as in the range of from 10-55 mg. Examples of the weight of the film matrix include weights of about 15 mg, about 20 mg, about 25 mg, about 30 mg, about 40 mg, about 50 mg or about 55 mg.

In an interesting embodiment of the invention, the unit dosage form further comprises an absorption enhancer. Absorption enhancers have demonstrated their effectiveness in delivering e.g. high molecular weight drugs, such as peptides, that generally exhibit low buccal absorption rates. Such absorption enhancers may act by a number of mechanisms, such a increasing the fluidity of the cell membrane, extracting inter/intracellular lipids, altering cellular proteins or altering surface mucin. The most commonly used absorption enhancers include azone, fatty acids, bile salts and surfactants, such as sodium dodecyl sulfate. Specific examples of absorption enhancers include, but are not limited to, 2,3-lauryl ether, aprotinin, azone, benzalkonium chloride, cetylpyridinium chloride, cetyltrimethyl ammonium bromide, cyclodextrin, dextran sulfate, glycol, lauric acid, lysophosphatidylcholine, menthol, phosphatidylcholine, polyoxyethylene, polysorbate 80, polyoxyethylene, phosphatidylcholine, sodium EDTA, sodium glycocholate, sodium glycodeoxycholate, sodium lauryl sulfate, sodium dodecyl sulfate, sodium salicylate, sodium taurocholate and sodium taurodeoxycholate, sulfoxides. The absorption enhancer is typically incorporated in the film matrix in an amount corresponding to 0.1-50% by weight of the film matrix, such as 1-20% by weight of the film matrix, e.g. 1-10% by weight of the film matrix. The absorption enhancer is typically comprised in the film matrix, i.e. the absorption enhancer is typically dissolved or dispersed in the film matrix.

In addition to the water-soluble matrix polymer and a ER-β selective agonist (and optionally one or more absorption enhancer(s)), the unit dosage form of the invention may include a variety of various auxiliary components, such as taste-masking agents; organoleptic agents, such as sweeteners and flavours, anti- and de-foaming agents; plasticizing agents; surfactants; emulsifying agents; thickening agents; binding agents; cooling agents; saliva-stimulating agents, such as menthol; antimicrobial agents; colorants; etc. Such various auxiliary components are comprised in the film matrix and is typically dissolved or dispersed in the film matrix.

Suitable sweeteners include both natural and artificial sweeteners. Specific examples of suitable sweeteners include, e.g.:

a) water-soluble sweetening agents such as sugar alcohols, monosaccharides, disaccharides, oligosaccharides and polysaccharides such as maltit, xylit, mannit, sorbit, xylose, ribose, glucose (dextrose), mannose, galactose, fructose (levulose), sucrose (sugar), maltose, invert sugar (a mixture of fructose and glucose derived from sucrose), partially hydrolyzed starch, corn syrup solids, dihydrochalcones, monellin, steviosides, and glycyrrhizin;
b) water-soluble artificial sweeteners such as the soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (acesulfame-K), the free acid form of saccharin, and the like;
c) dipeptide-based sweeteners, such as L-aspartic acid derived sweeteners, such as L-aspartyl-L-phenylalanine methyl ester (aspartame), L-alpha-aspartyl-N-(2, 2,4,4 5 tetramethyl-3-thietanyl)-D-alaninamide hydrate, methyl esters of L-aspartyl-L phenylglycerin and L-aspartyl-L-2,5, dihydrophenylglycine, L-aspartyl-2,5-dihydro-L phenylalanine, L-aspartyl-L-(1-cyclohexyen)-alanine, and the like;
d) water-soluble sweeteners derived from naturally occurring water-soluble sweeteners, such as a chlorinated derivatives of ordinary sugar (sucrose), known, for example, under the product description of Sucralose®; and
e) protein-based sweeteners such as thaurnatoccous danielli (Thaurnatin I and II).

In general, an effective amount of sweetener is utilised to provide the level of sweetness desired for a particular composition, and this amount will vary with the sweetener selected. This amount will normally be from about 0.01% to about 20% by weight, particularly from about 0.05% to about 10% by weight, of the film matrix. These amounts may be used to achieve a desired level of sweetness independent from the flavour level achieved from any optional flavour oils used.

Useful flavours (or flavouring agents) include natural and artificial flavours. These flavourings may be chosen from synthetic flavour oils and flavouring aromatics, and/or oils, oleo resins and extracts derived from plants, leaves, flowers, fruits and so forth, and combinations thereof. Non-limiting examples of flavour oils include: spearmint oil, cinnamon oil, peppermint oil, clove oil, bay oil, thyme oil, cedar leaf oil, oil of nutmeg, oil of sage, and oil of bitter almonds. Also useful are artificial, natural or synthetic fruit flavours such as vanilla, chocolate, coffee, cocoa and citrus oil, including lemon, orange, grape, lime and grapefruit, and fruit essences including apple, pear, peach, strawberry, raspberry, cherry, plum, pineapple, apricot and the like. These flavourings can be used individually or in combination. Commonly used flavours include mints such as peppermint, artificial vanilla, cinnamon derivatives, and various fruit flavours, whether employed individually or in combination. Flavourings such as aldehydes and esters including cinnamylacetate, cinnamaldehyde, citral, diethylacetal, dihydrocarvyl acetate, eugenyl formate, p-methylanisole, and the like may also be used. Further examples of aldehyde flavourings include, but are not limited to acetaldehyde (apple); benzaldehyde (cherry, almond); cinnamicaldehyde (cinnamon); citral, i.e., alpha citral (lemon, lime); neral, i.e. beta citral (lemon, lime); decanal (orange, lemon); ethyl vanillin (vanilla, cream); heliotropine, i.e., piperonal (vanilla, cream); vanillin (vanilla, cream); alpha-amyl cinnamaldehyde (spicy fruity flavours); butyraldehyde (butter, cheese); valeraldehyde (butter, cheese); citronellal (modified, many types); decanal (citrus fruits); aldehyde C-8 (citrus fruits); aldehyde C-9 (citrus fruits); aldehyde C-12 (citrus fruits); 2-ethyl butyraldehyde (berry fruits); hexenal, i.e. trans-2 (berry fruits); tolyl aldehyde (cherry, almond); veratraldehyde (vanilla); 12,6-dimethyl-5-heptenal, i.e. melonal (melon); 2-dimethyloctanal (greenfruit); and 2-dodecenal (citrus, mandarin); cherry; grape; essential oils, like menthol; mixtures thereof; and the like.

The amount of flavouring employed is normally a matter of preference, subject to such factors as flavour type, individual flavour, and strength desired. The amount may be varied in order to obtain the result desired in the final product. Such variations are within the capabilities of those skilled in the art without the need for undue experimentation. In general, amounts from about 0.01% to about 10% by weight of the film matrix are employed.

As discussed above, the unit dosage form may also include an anti-foaming and/or de-foaming agent, such as simethicone, which is a combination of a polymethylsiloxane and silicon dioxide. Simethicone acts as either an anti-foaming or de-foaming agent which reduces or eliminates air from the film composition. Anti-foaming agents will aid in preventing the introduction of air into the composition, while de-foaming agents will aid removing air from the composition.

The unit dosage form may be prepared and adhered to a second layer, i.e. a support or backing layer (liner) from which it is removed prior to use, i.e. before being introduced into the oral cavity. Preferably, the support or backing material is not water-soluble and may preferably consist of polyethylene-terephthalate, or other suitable materials well known to the skilled person. If an adhesive is used, it should preferably be a food grade adhesive that is not ingestible and does not alter the properties of the active ingredient(s).

In another embodiment of the invention, the unit dosage form of the invention further comprises another active drug substance, such as a progestin. The active drug substance is typically comprised in the film matrix.

Accordingly, in a more general aspect, the present invention relates to a unit dosage form comprising a thin water-soluble film matrix, wherein
a) said film matrix comprises at least one water-soluble matrix polymer;
b) said film matrix comprises an Estrogen Receptor beta (ERbeta) selective agonist, particularly a 8β- or 9α-substituted oestra-1,3,5(10)-triene as a Estrogen Receptor beta (ERbeta) selective agonist, in particular a ERbeta selective agonist chosen from the compounds:
9α-Vinyl-estra-1,3,5(10)-triene-3,16α-diol,
17β-Fluoro-9α-vinyl-estra-1,3,5(10)-triene-3,16α-diol,
18a-Homo-9α-vinyl-estra-1,3,5(10)-triene-3,16α-diol,
16α-Fluor-8β-vinyl-estra-1,3,5(10)-triene-3,17α-diol,
8β-vinyl-16α-fluoro-estra-1,3,5(10)-triene-3,17β-diol,
16β-Fluoro-8β-vinyl-estra-1,3,5(10)-triene-3,17β-diol,
8β-vinyl-estra-1,3,5(10)-triene-3,17β-diol, or derivatives thereof; and
   a progestin, particularly a 16,17 carbolactone derivative for example drospirenone or progestin selected from the group comprising: levonorgestrel, norgestrel, norethindrone (norethisterone), dienogest, norethindrone (norethisterone) acetate, ethynodiol diacetate, dydrogesterone, medroxyprogesterone acetate, norethynodrel, allylestrenol, lynestrenol, quingestanol acetate, medrogestone, norgestrienone, dimethisterone, ethisterone, chlormadinone acetate, megestrol, promegestone, desogestrel, 3-keto-desogestrel, norgestimate, gestodene, tibolone, cyproterone acetate.
c) said film matrix has a thickness of less than 300 μm.

The above unit dosage form comprising an Estrogen Receptor beta (ERbeta) selective agonist and a progestin can be used in cases where an opposing treatment is required.

However in view of the reduced inter-individual variability of serum levels achieved with the unit dosage form of the invention the selectivity of the wanted therapeutic ER-β mediated activity over the unwanted ER-α mediated endometrium-stimulating activity is obtained.

It should be understood, however, that in an interesting embodiment of the invention, the unit dosage form of the invention does not contain a progestin. Accordingly, in another interesting embodiment of the invention, the ER-β selective agonist, or a derivative thereof, is the only or sole therapeutically active drug substance present in the unit dosage form.

### Manufacture

The unit dosage form of the invention may be prepared by methods well known to the pharmaceutical technologist.

Typically, a drug solution is prepared by dissolving the ER-β selective agonist, or a derivatives thereof, in an appropriate solvent. The solvent is commonly a relatively volatile solvent, such as an alcohol, in particular ethanol. A matrix polymer solution is then prepared by adding the water-soluble matrix polymer to a suitable solvent, such as alcohol or a mixture of an alcohol and water. Particularly, the solvent is an ethanol/water mixture. As will be understood, the time and conditions needed to dissolve the water-soluble matrix polymer will depend on the polymer and the solvent used. Thus, in some cases the water-soluble matrix polymer may dissolve easily at room temperature and with only gentle stirring, while in other cases it will be necessary to apply heat and vigorous stirring to the system. In a typical embodiment, the mixture is stirred for 1-4 hours, preferably for about 2 hours, or until a solution is obtained. The solution is typically stirred at a temperature of 60-80° C., such as about 70° C. After cooling to room temperature, the drug solution is poured into the matrix polymer solution and mixed thoroughly. The resulting solution (coating solution) can be used for coating immediately or within a few days, commonly within one day. The various amounts of solvent, matrix polymer, etc. are adjusted to reach a solid content of the coating solution of about 5-50% by weight, preferably 10-40% by weight, in particular 20-35% by weight.

In an alternative embodiment, the coating solution may be prepared directly by adding the ER-β selective agonist, or a derivatives thereof, to an appropriate solvent, preferably an alcohol, in particular ethanol, followed by addition of water and subsequent addition of the matrix polymer. The mixture is then processed as described above until a solution is obtained. The resulting solution (coating solution) can be used for coating immediately or within a few days, commonly within one day. The various amounts of solvent, matrix polymer, etc. are adjusted to reach a solid content of the coating solution of about 5-50% by weight, preferably 10-40% by weight, in particular 20-35% by weight.

In an alternative embodiment, the coating solution may be prepared by directly adding the ER-β selective agonist, or a derivative thereof, to an appropriate polymer solution and dissolving the drug in said polymer solution. In this case the polymer solution is prepared beforehand by dissolving the polymer in the solvent/water mixture according the above described process. After dissolution of the active component in the polymer solution, the resulting solution (coating solution) can be used for coating immediately or within a few days, commonly within one day. The various amounts of solvent, matrix polymer, etc. are adjusted to reach a solid content of the coating solution of about 5-50% by weight, preferably 10-40% by weight, in particular 20-35% by weight.

Other excipients, auxiliary components and/or active drug substances may be added during any of the above mentioned steps.

If needed, the coating solution is degassed before being spread out on a suitable support or backing layer (liner). Examples of suitable liners include, but are not limited to polyethylene-terephthalate (PET) liners, such as Perlasic® LF75 (available from Perlen Converting), Loparex® LF2000 (available from Loparex BV) and Scotchpack® 9742 (available from 3M Drug delivery Systems). In one embodiment of the invention, the coating solution is spread out with the aid of a spreading box onto a suitable liner and dried for 12-24 hours at room temperature. A thin film of 30-100 μm thickness, preferably 40-80 μm thickness is then produced, which is subsequently cut into pieces of the desired size and shape. Alternatively, the coating solution is coated as a thin film onto a suitable liner and in-line dried using an automated coating and drying equipment (e.g. by Coatema Coating Machinery GmbH, Dormagen, Germany) using a drying temperature of 40-120° C. A thin transparent film is then obtained, which is subsequently cut or punched into pieces of the desired size and shape. Said film is transparent, translucent or opaque.

### Therapeutic Use and Administration

It will be understood that the unit dosage form of the invention is administered intraorally, i.e. the unit dosage form is administered to the oral cavity and the active drug substance is subsequently absorbed via one or more of the oral mucosae. Thus, the active ingredient is suitable for lingual administration, sublingual administration, buccal administration and palatal administration.

Accordingly, in another aspect, the present invention relates to a unit dosage form of the invention for use as a medicament.

In yet another aspect, the present invention relates to a unit dosage form of the invention for treating, alleviating or preventing a physical condition in a female mammal caused by insufficient endogenous levels of estrogen, such as vasomotor symptoms (particularly hot flashes and night sweats), symptoms of urogenital atrophy, sleep disorders, memory problems, anxiety, depression and other mood disorders, decrease in bone mineral density, osteoporosis, or increased risk or incidence of bone fracture.

Deficient levels of estrogen may be caused by natural or surgical menopause, peri-menopause, primary ovarian failure, or a variety of other pathological conditions leading to pre-menopausal hypogonadism. Low levels of estrogen, irrespective of the cause, lead to an overall decreased quality of life for women. Symptoms, diseases and conditions range from merely being inconvenient to life threatening. The unit dosage form described herein provide effective alleviation of physiological and psychological signs of estrogen deficiency. Transient symptoms, such as vasomotor signs and psychological symptoms are certainly embodied with the realm of therapy.

Vasomotor symptoms comprise but are not limited to hot flushes, sweating attacks such as night sweats, and palpitations. The vasomotor symptoms may be “mild”, “moderate” or “severe” as defined by the FDA guidelines (cited supra). Psychological symptoms of estrogen deficiency comprise, but are not limited to, insomnia and other sleep disturbances, poor memory, loss of confidence, mood changes, anxiety, loss of libido, difficulties in concentration, difficulty in making decisions, diminished energy and drive, irritability and crying spells. The treatment or alleviation of the aforementioned symptoms can be associated with the peri-menopausal phase of a woman's life or after, sometimes long time after, menopause. It is anticipated that the unit dosage forms described herein are applicable to these and other transient symptoms during the peri-menopausal phase, menopause, or post-menopausal phase. Moreover, the aforementioned symptoms can be alleviated if the cause of the estrogen deficiency is hypogonadism, castration or primary ovarian failure. In another embodiment of the invention, the unit dosage forms described herein are used for the treatment or alleviation of permanent effects of estrogen deficiency. Permanent effects comprise physical changes such as urogenital atrophy, atrophy of the breasts, cardiovascular disease, changes in hair distribution, thickness of hair, changes in skin condition and osteoporosis. Urogenital atrophy, and conditions associated with it such as vaginal dryness, increase in vaginal pH and subsequent changes in flora, or events which lead to such atrophy, such as decreases in vascularity, fragmentation of elastic fibres, fusion of collagen fibres, or decreases in cell volume, are symptoms thought to be particularly relevant to be treated or alleviated with the unit dosage forms described herein. Furthermore, the unit dosage forms described herein are thought to be relevant to other urogenital changes associated with estrogen deficiency, decreases in mucus production, changes in cell population, decreases in glycogen production, decreases in growth of lactobacilli or increases in growth of streptococci, staphylococci, or coliform bacilli. Other associated changes that are thought to be preventable by administration of the unit dosage forms described herein are those that may render the vagina susceptible to injury or infection, such as exudative discharges, vaginitis, and dyspareunia. Furthermore, infections of the urinary tract and incontinence are other common symptoms associated with lowered estrogen levels. Other embodiments of the invention include the prevention or alleviation of physical changes associated with estrogen deficiency, such as changes in the skin, changes in hair distribution, thickness of hair, atrophy of the breasts, or osteoporosis. A particularly interesting embodiment of the invention is directed to lessening the frequency, persistence, duration and/or severity of hot flushes, sweating attacks, palpitations, sleep disturbances, mood changes, nervousness, anxiety, poor memory, loss of confidence, loss of libido, poor concentration, diminished energy, diminished drive, irritability, urogenital atrophy, atrophy of the breasts, cardiovascular disease, changes in hair distribution, thickness of hair, changes in skin condition and osteoporosis (including prevention of osteoporosis), most notably hot flushes, sweating attacks, palpitations, sleep disturbances, mood changes, nervousness, anxiety, urogenital atrophy, atrophy of the breasts, as well as prevention or management of osteoporosis. Another interesting embodiment of the invention is directed to treatment or alleviation of hot flushes, sweating attacks, palpitations, sleep disturbances, mood changes, nervousness, anxiety, poor memory, loss of confidence, loss of libido, poor concentration, diminished energy, diminished drive, irritability, urogenital atrophy, atrophy of the breasts, cardiovascular disease, changes in hair distribution, thickness of hair, changes in skin condition and osteoporosis (including prevention of osteoporosis), most notably hot flushes, sweating attacks, palpitations, sleep disturbances, mood changes, nervousness, anxiety, urogenital atrophy, atrophy of the breasts, as well as prevention or management of osteoporosis.

In a preferred embodiment of the invention, the female mammal to be treated according to the invention is a woman, in particular a postmenopausal woman.

The terms “pre-menopause”, “peri-menopause”, “menopause” and “post-menopause” are used in their conventional meaning, e.g. as defined on page 9 of “The Controversial Climacteric”; P. A. van Keep et al. Ed., MTP Press (1981). More particularly, the term “menopause” is understood as the last natural (ovary-induced) menstruation. It is a single event and a result of an age-dependent dysfunction of the ovarian follicles. Menopause results from the ovaries decreasing their production of the sex hormones estrogen and progesterone. When the number of follicles falls below a certain threshold, the ovaries can no longer produce mature follicles and sex hormones. The ability to reproduce ends with menopause. The peri-menopausal phase begins with the onset of climacteric symptoms when the cycle becomes irregular and ends one year after menopause. The end of peri-menopausal phase can be identified after a protracted period of time without bleeding. Post-menopause is the phase that begins at menopause and continues until death.

In a further, and particular preferred embodiment of the invention, the postmenopausal woman to be treated according to the invention is a hysterectomised postmenopausal woman.

Hysterectomy is the surgical removal of the uterus. A total hysterectomy is removal of the uterus and cervix. A partial hysterectomy is removal of the uterus leaving the stump of the cervix (also called supra-cervical). Hysterectomy can be accompanied by surgical removal of the ovaries (oophorectomy). Removal of the female gonads, the ovaries, is female castration. Women who undergo total hysterectomy with bilateral salpingo-oophorectomy (removal of both ovaries, i.e. castration) lose most of their hormone production, including many estrogens and progestins. A woman who is undergoing natural menopause has intact and functional female organs, while a woman who has been hysterectomised and castrated does not. Accordingly, in the present context the term “hysterectomised woman” refers to a woman who has undergone total or partly hysterectomy.

The unit dosage forms of the present invention have a considerable higher bioavailability than orally administered tablets. Thus, a bioavailability of more than 30% will typically be achieved. More particularly, a bioavailability in the range of from 30-100%, such as 40-90% will typically be achieved. In an interesting embodiment of the invention, a bioavailability of more than 50%, particularly more than 60% is achieved. This, in turn, has the consequence that therapeutic effective serum levels of an ER-β agonist can be achieved although a significantly lower dose of the ER-β agonist is administered as compared to oral administration. Evidently, the achieved bioavailability as well as the serum level of ER-β agonist will be dependent on the actual design of the unit dosage form of the invention as well as the drug load and the applied ER-β selective agonist or derivatives thereof.

### EXPERIMENTAL

### Example 1

### Preparation of Wafers

### Preparation of the Coating Solution—Option A

A drug solution containing 0.75 g ER-β selective agonist is prepared by dissolving the drug in 236.7 g ethanol (96%) under stirring. A polymer solution is prepared by strewing 289.25 g Hydroxypropylcellulose (HPC) or Hydroxypropyl methylcellulose (HPMC) onto 473.3 g water. The HPC or HPMC dissolves after stirring for 1-2 hours at 70° C. After cooling to room temperature, the drug solution is poured into the polymer solution and mixed thoroughly. The resulting solution (coating solution) can be used for coating immediately or within a few days commonly within one day.

### Preparation of the Coating Solution—Option B

A coating solution is prepared by dissolving 0.75 g ER-β selective agonist in 236.7 g ethanol (96%) under stirring. After admixing with 473.3 g water, 289.25 g HPC or HPMC is added and dissolves after stirring for 2 hours at 70° C. The resulting solution (coating solution) can be used for coating immediately or within a few days, commonly within one day.

### Preparation of Wafers—Option 1

The coating solution is degassed and spread out with the aid of a spreading box onto a polyethylene-terephthalate (PET) liner (e.g. Scotchpak® 9742 or Perlasic® LF75) and dried for 24 hours at room temperature. A thin transparent film which is about 40 μm thick is produced. Wafers are obtained by punching out samples of 2-7 cm² size.

### Preparation of Wafers—Option 2

The coating solution is degassed and coated as a thin film onto a polyethylene-terephthalate (PET) liner (e.g. Scotchpak® 9742 or Perlasic® LF75) and in-line dried using an automated coating and drying equipment (Coatema Coating Machinery GmbH, Dormagen, Germany). A drying temperature of 40-120° C. is applied. A thin transparent film which is about 40 μm thick is produced. Wafers are obtained by punching out samples of 2-7 cm² size.

Using the above-mentioned manufacturing methods, wafers having the following composition were prepared:

ER-β Selective Agonist Wafer, 25 μg (with Hydroxypropyl Cellulose Matrix), 2 cm²

| Name of ingredient | Quantity | Function |
|---|---|---|
| Active ingredients | | |
| ER-β selective agonist | 0.025 mg | Active ingredient |
| Other ingredients | | |
| Hydroxypropyl cellulose | 9.975 mg | Matrix polymer |
| Purified water* | q.s. | Process solvent |
| Ethanol 96%* | q.s. | Process solvent |
| Total mass: | 10.000 mg | |

*evaporates during manufacturing

ER-β Selective Agonist Wafer, 62.5 μg (with Hydroxypropyl Cellulose Matrix), 5 cm²

| Name of ingredient | Quantity | Function |
|---|---|---|
| Active ingredients | | |
| ER-β selective agonist | 0.0625 mg | Active ingredient |
| Other ingredients | | |
| Hydroxypropyl cellulose | 24.9375 mg | Matrix polymer |
| Purified water* | q.s. | Process solvent |
| Ethanol 96%* | q.s. | Process solvent |
| Total mass: | 25.000 mg | |

*evaporates during manufacturing

ER-β Selective Agonist Wafer, 150 μg (with Hydroxypropyl Cellulose Matrix), 3 cm²

| Name of ingredient | Quantity | Function |
|---|---|---|
| Active ingredients | | |
| ER-β selective agonist | 0.150 mg | Active ingredient |
| Other ingredients | | |
| Hydroxypropyl cellulose | 14.850 mg | Matrix polymer |
| Purified water* | q.s. | Process solvent |
| Ethanol 96%* | q.s. | Process solvent |
| Total mass: | 15.000 mg | |

*evaporates during manufacturing

ER-β Selective Agonist Wafer, 250 μg (with Hydroxypropyl Cellulose Matrix), 5 cm²

| Name of ingredient | Quantity | Function |
|---|---|---|
| Active ingredients | | |
| ER-β selective agonist | 0.250 mg | Active ingredient |
| Other ingredients | | |
| Hydroxypropyl cellulose | 24.750 mg | Matrix polymer |
| Purified water* | q.s. | Process solvent |
| Ethanol 96%* | q.s. | Process solvent |
| Total mass: | 25.000 mg | |

*evaporates during manufacturing

ER-β Selective Agonist Wafer, 625 μg (with Hydroxypropyl Cellulose Matrix), 5 cm²

| Name of ingredient | Quantity | Function |
|---|---|---|
| Active ingredients | | |
| ER-β selective agonist | 0.625 mg | Active ingredient |
| Other ingredients | | |
| Hydroxypropyl cellulose | 24.375 mg | Matrix polymer |
| Purified water* | q.s. | Process solvent |
| Ethanol 96%* | q.s. | Process solvent |
| Total mass: | 25.000 mg | |

*evaporates during manufacturing

ER-β Selective Agonist Wafer, 875 μg (with Hydroxypropyl Cellulose Matrix), 7 cm²

| Name of ingredient | Quantity | Function |
|---|---|---|
| Active ingredients | | |
| ER-β selective agonist | 0.875 mg | Active ingredient |
| Other ingredients | | |
| Hydroxypropyl cellulose | 34.125 mg | Matrix polymer |
| Purified water* | q.s. | Process solvent |
| Ethanol 96%* | q.s. | Process solvent |
| Total mass: | 35.000 mg | |

*evaporates during manufacturing

ER-β Selective Agonist Wafer, 1000 μg (with Hydroxypropyl Cellulose Matrix), 5 cm²

| Name of ingredient | Quantity | Function |
|---|---|---|
| Active ingredients | | |
| ER-β selective agonist | 1.000 mg | Active ingredient |
| Other ingredients | | |
| Hydroxypropyl cellulose | 24.000 mg | Matrix polymer |
| Purified water* | q.s. | Process solvent |
| Ethanol 96%* | q.s. | Process solvent |
| Total mass: | 25.000 mg | |

*evaporates during manufacturing

ER-β Selective Agonist Wafer, 1500 μg (with Hydroxypropyl Cellulose Matrix), 7 cm²

| Name of ingredient | Quantity | Function |
|---|---|---|
| Active ingredients | | |
| ER-β selective agonist | 1.500 mg | Active ingredient |
| Other ingredients | | |
| Hydroxypropyl cellulose | 36.000 mg | Matrix polymer |
| Purified water* | q.s. | Process solvent |
| Ethanol 96%* | q.s. | Process solvent |
| Total mass: | 37.500 mg | |

*evaporates during manufacturing

ER-β Selective Agonist Wafer, 2000 μg (with Hydroxypropyl Cellulose Matrix). 5 cm²

| Name of ingredient | Quantity | Function |
|---|---|---|
| Active ingredients | | |
| ER-β selective agonist | 2.000 mg | Active ingredient |
| Other ingredients | | |
| Hydroxypropyl cellulose | 48.000 mg | Matrix polymer |
| Purified water* | q.s. | Process solvent |
| Ethanol 96%* | q.s. | Process solvent |
| Total mass: | 50.000 mg | |

*evaporates during manufacturing

ER-β Selective Agonist Wafer, 2500 μg (with Hydroxypropyl Cellulose Matrix). 7 cm²

| Name of ingredient | Quantity | Function |
|---|---|---|
| Active ingredients | | |
| ER-β selective agonist | 2.500 mg | Active ingredient |
| Other ingredients | | |
| Hydroxypropyl cellulose | 47.500 mg | Matrix polymer |
| Purified water* | q.s. | Process solvent |
| Ethanol 96%* | q.s. | Process solvent |
| Total mass: | 50.000 mg | |

*evaporates during manufacturing

ER-β Selective Agonist Wafer, 3000 μg (with Hydroxypropyl Cellulose Matrix), 7 cm²

| Name of ingredient | Quantity | Function |
|---|---|---|
| Active ingredients | | |
| ER-β selective agonist | 3.000 mg | Active ingredient |
| Other ingredients | | |
| Hydroxypropyl cellulose | 57.000 mg | Matrix polymer |
| Purified water* | q.s. | Process solvent |
| Ethanol 96%* | q.s. | Process solvent |
| Total mass: | 60.000 mg | |

*evaporates during manufacturing

ER-β Selective Agonist Wafer, 250 μg (with Hydroxypropyl Methylcellulose Matrix), 5 cm²

| Name of ingredient | Quantity | Function |
|---|---|---|
| Active ingredients | | |
| ER-β selective agonist | 0.250 mg | Active ingredient |
| Other ingredients | | |
| Hydroxypropyl | 24.750 mg | Matrix polymer |
| methylcellulose (HPMC) | | |
| Purified water* | q.s. | Process solvent |
| Ethanol 96%* | q.s. | Process solvent |
| Total mass: | 25.000 mg | |

*evaporates during manufacturing

ER-β Selective Agonist Wafer, 625 μg (with Hydroxypropyl Methylcellulose Matrix), 5 cm²

| Name of ingredient | Quantity | Function |
|---|---|---|
| Active ingredients | | |
| ER-β selective agonist | 0.625 mg | Active ingredient |
| Other ingredients | | |
| Hydroxypropyl | 24.375 mg | Matrix polymer |
| methylcellulose (HPMC) | | |
| Purified water* | q.s. | Process solvent |
| Ethanol 96%* | q.s. | Process solvent |
| Total mass: | 25.000 mg | |

*evaporates during manufacturing

As will be understood, analogous wafers which contain other amounts of ER-α selective agonist and/or which contain ER-β selective agonist derivatives can easily be manufactured using the procedures described herein.

Furthermore, the above ER-β selective agonist wafer formulations were prepared using the compound 17β-Fluoro-9α-vinyl-estra-1,3,5(10)-triene-3,16α-diol which should be considered as a non-limiting example of a an Estrogen Receptor beta (ER-β) selective agonist to be used in the unit dosage form according to the invention.

In the present application, any amount given in percentage (%) should be intended as percentage by weight (% w/w) if not differently specified.

### Example 2

### Clinical Trial (PK Study)

### 1. Study Outline

| | |
|---|---|
| Study objective | To investigate the pharmacokinetic properties |
| | and general safety and tolerability of different |
| | doses of an ER-β selective agonist in two |
| | different polymer matrices as wafer formulations |
| | at two application sites |
| Study design | Single-center open-label, sequential design |
| Study population | Healthy postmenopausal women, aged 45-75 |
| | years |
| Treatment | Two different ER-β selective agonist wafer |
| | formulations |
| | Three different ER-β selective agonist wafer |
| | doses |
| Duration | Up to three months with 2 study parts |
| | evaluating three single-dose treatments each |
| | with at least one week washout between each |
| | treatment |
| Variables | Primary: Pharmacokinetic standard variables |
| | e.g. Area Under the Curve (AUC), maximum |
| | drug concentration (Cmax), terminal half-life |
| | Secondary: ECG, blood pressure, pulse rate, |
| | standard laboratory parameters, questionnaire |
| | regarding irritability and tolerability at |
| | application site, adverse events |

### Preliminary Results:

12 postmenopausal women completed the first part of the study and preliminary results are available. Each woman received sequentially three doses of a wafer formulation of an ER-β selective agonist. Compared to a tablet formulation, the wafer formulation resulted in a considerably higher bioavailability and significantly reduced inter-individual variability with respect to drug serum levels. A dose proportional increase of serum levels as measured by AUC and Cmaxwas observed in the evaluated dose range.

### Conclusions:

From the preliminary data with the wafer dosage form, the inventors conclude that therapeutically meaningful drug levels of ER-β selective agonist can be achieved with a much lower drug load than with other administration forms, e.g. oral tablets. Furthermore, the reduced inter-individual variability with respect to serum levels will reduce the number of individuals in whom very high serum levels of the drug are achieved and is, therefore, resulting in a reduced incidence of potential drug related side effects. Since the inter-individual variability is smaller than the estimated therapeutic window between wanted ER-β mediated effects and unwanted ER-α mediated stimulation of the endometrium the omission of progestin co-administration in women with uterus appears to be feasible, thus avoiding progestin related side effects like uterine bleeding, breast pain, breast tenderness, mood disturbances, and potential long-term progestin-associated safety risks.

### Example 3

### In Vivo Evaluation of Placebo Wafers

Placebo wafer formulations of the polymer matrices, containing also additives (e.g. stabilizers, plasticizers) were evaluated in a human test panel (n=8) with respect to handling and administration. For both properties following features were evaluated:

| | |
|---|---|
| Handling: | film thickness |
| | flexibility |
| Administration: | disintegration |
| | adherence (to palate) |
| | taste |

Especially the taste and disintegration time seem to be most relevant parameters for the acceptability of the formulation for long term use.

The film thickness and flexibility were additionally quantified and correlated to the in-vivo evaluation.

The film thickness was measured by a MiniTest 600, Erichsen, Hemer, Germany The mechanical properties were quantified by measurement of the tensile strength and elongation (Zwick Material Testing, Ulm, Germany) and calculation of the modulus of elasticity, E, by following equation:

$$E = \frac{tensile strength}{elongation} = \frac{F / A}{Δ L / {L}_{0}}$$

with
E: modulus of elasticity (Young's modulus)
F: force (in N) applied to the object
A₀: original cross-sectional area through which the force is applied
ΔL: amount by which the length of the object changes
L₀: original length of the object

### Evaluation of Film Thickness of Placebo Wafer Formulation

| | Film | | | |
|---|---|---|---|---|
| Polymers/ | thickness, | | | |
| Additives | μm | Too thick | ok | Too thin |
| HPMC | 68 ± 3 | 0 | 8 | 0 |
| HPMC + PG | 52 ± 4 | 0 | 5 | 3 |
| HPC (Klucel ® EF) | 46 ± 2 | 0 | 4 | 4 |
| HPMC + TEC | 51 ± 3 | 0 | 6 | 2 |
| HPMC + gamma CD | 81 ± 10 | 5 | 3 | 0 |
| HPMC + Propylgallat | 51 ± 4 | 1 | 7 | 0 |

### Evaluation of Flexibility of Placebo Wafer Formulation

| | +++ | | |
|---|---|---|---|
| Polymers/ | (in all | ++ | + |
| Additives | directions) | (90-180°) | (<90°) |
| HPMC | 6 | 2 | 0 |
| HPMC + PG | 5 | 3 | 0 |
| HPC (Klucel ® EF) | 8 | 0 | 0 |
| HPMC + TEC | 7 | 1 | 0 |
| HPMC + gamma | 0 | 6 | 2 |
| CD | | | |
| HPMC + | 6 | 2 | 0 |
| Propylgallat | | | |

### Determination of the Mechanical Properties and Modulus of Elasticity of Placebo Wafer Formulation

| Polymers/ | | | E, |
|---|---|---|---|
| Additives | F, N | ΔL/L₀, % | MPa |
| HPMC | 39.1 ± 3.0 | 10.8 ± 2.3 | 520 ± 71 |
| HPMC + PG | 25.8 ± 0.9 | 9.3 ± 0.3 | 447 ± 12 |
| HPC (Klucel ® EF) | 6.3 ± 0.8 | 23.9 ± 8.1 | 51 ± 12 |
| HPMC + TEC | 15.5 ± 2.1 | 6.6 ± 0.9 | 385 ± 4 |
| HPMC + gamma | 37.6 ± 3.1 | 9.3 ± 0.7 | 414 ± 14 |
| CD | | | |
| HPMC + | 35.9 ± 1.7 | 10.6 ± 0.7 | 554 ± 35 |
| Propylgallat | | | |

For the unit dosage forms described in the present “Example 3” the Polymer+Additives compositions were:
HPMC+20% PG (propylene glycol)
HPMC+20% TEC (triethylcitrate)
HPMC+5% gamma CD (gamma-Cyclodextrin)
HPMC+2.3% Propylgallat

In the present application, any amount given in percentage (%) should be intended as percentage by weight (% w/w). The amount of additive contained is given based on the total formulation.

The polymer and the additive were dissolved in ethanol/water 2:1 to receive the coating solution. Placebo wafers were manufactured from this coating solution according the above described procedure “preparation of wafers—option 1”.

Using HPC as a polymer matrix the resulting wafers were much more flexible than wafers containing HPMC as a polymer matrix, even those containing plasticizers (e.g. propylene glycol (PG) or triethylcitrate (TEC)). The measurement of the mechanical properties confirmed, that the modulus of elasticity was strongly decreased and the %-elongation (ΔL/L₀) much increased for HPC wafers compared to all other formulations.

Disintegration Time of Placebo Wafer Formulation after Administration (Normalized to a Wafer Thickness of 50 μm)

| Polymers/ | |
|---|---|
| Additives | Time, s |
| HPMC | 18.7 |
| HPMC + PG | 14.2 |
| HPC (Klucel ® EF) | 28.8 |
| HPMC + TEC | 19.0 |
| HPMC + gamma CD | 17.7 |
| HPMC + Propylgallat | 19.1 |

The mean value for the time until complete disintegration of the wafers was 20.3 seconds (S.D.: ±5.3 seconds). Additions of relevant amounts of liquid additives (e.g. plasticizers) resulted in a decrease of the disintegration time (e.g. HPMC vs. HMPC+PG). However, some polymers also prolonged the disintegration time remarkably (e.g. HPC).

Adherence to the Palate of Placebo Wafer Formulation after Administration

| Polymers/ | | | |
|---|---|---|---|
| Additives | Very good | good | low |
| HPMC | 1 | 4 | 3 |
| HPMC + PG* | 3 | 4 | 0 |
| HPC (Klucel ® EF) | 6 | 2 | 0 |
| HPMC + TEC* | 2 | 5 | 0 |
| HPMC + gamma CD | 1 | 6 | 1 |
| HPMC + Propylgallat | 2 | 4 | 2 |

*n = 7

Taste of Placebo Wafer Formulation after Administration

| Polymers/ | | tolerable | | in- |
|---|---|---|---|---|
| Additives | enjoyable | (neutral) | bad | acceptable |
| HPMC | 1 | 5 | 2 | 0 |
| HPMC + PG | 0 | 6 | 2 | 0 |
| HPC (Klucel ® EF) | 3 | 6 | 0 | 0 |
| HPMC + TEC | 0 | 0 | 1 | 7 |
| HPMC + gamma CD | 0 | 2 | 6 | 0 |
| HPMC + Propylgallat | 0 | 4 | 4 | 0 |

In general the taste of the formulation was related to the polymer matrix.

Most additives altered the taste of the formulations significantly such that the taste turned bad, or even inacceptable (e.g. Triethylcitrate (TEC), gamma-Cyclodextrin (gamma CD)).

### Overview of Results of the In Vivo Evaluation of Placebo Wafer Formulations

| Polymers/ | | | Disintegration | | |
|---|---|---|---|---|---|
| Additives | Flexibility | Stickiness | time | Adherence | Taste |
| HPMC | + | + | + | − | + |
| HPMC + PG | + | − | ↓ | + | + |
| HPC | ++ | − | ↑ | ++ | +++ |
| (Klucel ® | | | | | |
| EF) | | | | | |
| HPMC + | + | + | + | + | −− |
| TEC | | | | | |
| HPMC + | − | + | + | + | − |
| gamma CD | | | | | |
| HPMC + | + | + | + | + | + |
| Propylgallat | | | | | |

The overall evaluation of the results revealed surprisingly a correlation of the perceived taste of the formulations to their flexibility. Thus, the flexibility of the film seems to have a crucial impact on the perceivable taste.

Moreover, also the adherence to the palate was improved with improved flexibility of the formulations.

In conclusion, more flexible films will result therefore in wafers with higher acceptability by the patients due to a higher comfort during administration related to an improved taste and adherence to the mucosa.

The present wafer demostrates improved mouthfeel and taste, by defining favourable thickness and elasticity, able to confer improved patient acceptability. this particularly the case with the film matrix having a modulus of elasticity <200 MPas or particularly <150 MPas or more particularly <100 MPas and a %-elongation >15%, or particularly >20%.

1. A unit dosage form comprising a thin water-soluble film matrix, wherein
a) said film matrix comprises at least one water-soluble matrix polymer;
b) said film matrix comprises a Estrogen Receptor beta (ER-β) selective agonist, or a derivative thereof; and
c) said film matrix has a thickness of less than 300 μm.

2. A unit dosage according to claim 1 wherein said film matrix comprises a 8β- or 9α-substituted oestra-1,3,5(10)-triene as ER-β selective agonist, or a derivative thereof.

3. The dosage form according to claim 1, wherein said ER-β selective agonist is one of the following compounds:
9α-Vinyl-estra-1,3,5(10)-triene-3,16α-diol,
17β-Fluoro-9α-vinyl-estra-1,3,5(10)-triene-3,16α-diol,
18a-Homo-9α-vinyl-estra-1,3,5(10)-triene-3,16α-diol,
16α-Fluor-8β-vinyl-estra-1,3,5(10)-triene-3,17α-diol,
8β-vinyl-16α-fluoro-estra-1,3,5(10)-triene-3,17β-diol,
16β-Fluoro-8β-vinyl-estra-1,3,5(10)-triene-3,17β-diol,
8β-vinyl-estra-1,3,5(10)-triene-3,17β-diol,
or derivatives thereof.

4. The dosage form according to claim 1, wherein said water-soluble matrix polymer is selected from the group consisting of a cellulosic material, a synthetic polymer, a gum, a protein, a starch, a glucan and mixtures thereof.

5. The dosage form according to claim 2, wherein said cellulosic material is selected from the group consisting of carboxymethyl cellulose, methyl cellulose, ethyl cellulose, hydroxymethyl cellulose, hydroxyethyl cellulose, hydroxypropyl cellulose, hydroxymethylpropyl cellulose and hydroxypropylmethyl cellulose.

6. The dosage form according to claim 1, wherein said film matrix comprises up to 5000 μg, preferably up to 3000 μg, most preferably up to 1500 μg of a ER-β selective agonist, or derivatives thereof.

7. The dosage form according to claim 1, wherein said film matrix has a thickness of less than 200 μm, or of less than 100 μm.

8. The dosage form according to claim 1, wherein said film matrix has a surface area of 2-10 cm², or of 3-7 cm², or of 4-6 cm².

9. The dosage form according to claim 1, wherein said film matrix has a weight in the range of from 5-200 mg, or in the range of from 10-100 mg, or in the range of from 10-50 mg.

10. The dosage form according to claim 1, wherein said film matrix has a modulus of elasticity <200 MPas or <150 MPas or <100 MPas.

11. The dosage form according to claim 1, wherein said film matrix has a %-elongation >15%, or >20%.

12. The dosage form according to claim 1, wherein said dosage form comprises an absorption enhancer.

13. The dosage form according to claim 11, wherein said absorption enhancer is dissolved or dispersed in the film matrix.

14. The dosage form according to claim 1, wherein said film matrix further comprises a progestin.

15. A unit dosage form as defined in claim 1 for use as a medicament.
